(19) United States
(12) Patent Application Publication
(10) Pub. No.: US 2023/0263929 A1
(43) Pub. Date: Aug. 24, 2023

(54) FILTER FOR SANITIZING AIR IN INDOOR ENVIRONMENTS

(71) Applicant: NANOHUB S.R.L., Milano (IT)

(72) Inventors: Stefano PERBONI, Milano (IT); Sudipto Kumar PAL, Milano (IT)

(73) Assignee: NANOHUB S.R.L., Milano (IT)

(21) Appl. No.: 18/009,569

(22) PCT Filed: Jun. 9, 2021
(86) PCT No.: PCT/EP2021/065516

§ 371 Date: Dec. 9, 2022

(30) Foreign Application Priority Data
Jun. 10, 2020 (IT) 102020000013921

(51) Int. Cl. A61L 9/20; B01J 35/00; B01J 23/30; B01J 35/04; D06M 11/83; D06M 16/00; B01D 53/88; B01D 53/00; F24F 8/167; F24F 8/108

(52) U.S. Cl. CPC A61L 9/205; B01D 53/007; B01D 53/885; B01J 23/30; B01J 35/004; B01J 35/04; D06M 11/83; D06M 16/00; F24F 8/108; F24F 8/167; A61L 2209/12; A61L 2209/14; A61L 2209/16; D06M 2101/06

(57) ABSTRACT

The filter according to the present invention is based on the combined action of two materials: tungsten trioxide (WO3), used for implementing the photocatalytic reactor, and a solution of copper (Cu) nanocluster. In a preferred embodiment of the present invention, these materials are applied to suitable supports (filters), one of a mesh/grid made of metal (or other material, e.g. plastics material) for the photocatalyst and the other of fabric made of cotton (or other hydrophilic material), for sanitizing the fluids by way of the use of said supports in systems for air treatment (devices which take the air from the environment and/or from the exterior, filter it, and reemit it after passage through the filtration system in question) and/or water filtration.

The present invention has high efficiency and is a significant innovation, including on the basis of the efficacy tests performed in the present period of health emergency, including on the infective virus COVID-19.

### TECHNICAL FIELD

The present invention relates to a method and system for filtering aeroliquid and liquid fluids and in particular for air in confined (indoor) environments, which makes it possible to sanitize it to reduce pollutants, VOCs, bacteria, viruses, spores, moulds and other organic and inorganic compounds harmful to the health of the people who live in the environments.

### TECHNICAL BACKGROUND

The quality of the air in closed environments (in the following also “indoor”) takes on, directly or indirectly, a leading role in the well-being of persons, representing one of the main determining factors for health, since often exposure to indoor pollutants is dominant over external (“outdoor”) exposure. The quality of indoor air depends not only on the quality of the outdoor air, but also on the presence of internal sources of emission and diffusion of contaminants having a concentration of chemical and biological pollutants which are capable of influencing the features thereof.

In particular, following the recent health emergency due to the spread of COVID-19 (SARS-CoV-2), a recent document from the Italian National Institute of Health (ISS) stated that, “In the face of the current national situation, which has led to the introduction of public health provisions (including measures to reduce contact and limitation on the circulation of persons and on leaving one’s own residence or domicile) necessary for preventing, impeding and delaying the spread of the epidemic of SARS-CoV-2, the virus which causes COVID-19, the quality of indoor air is taking on significant importance in protection, safeguarding and prevention for the health of citizens and of workers.

In the various buildings and environments in which a wide range of activities and functions take place (such as residences, offices, healthcare facilities, pharmacies, parapharmacies, banks, post offices, supermarkets, airports, stations and means of public transport), it is helpful to promote processes which make it possible to acquire preventative health behaviours and measures. In general, in any situation, appropriate standards of behaviour have an important role in improving the quality of indoor air and, in relation to containing or slowing the transmission of SARS-CoV-2, in the various environments, take on a particular significance and prominence.” (from ISS COVID-19 Report No. 5/2020).

This has resulted in increased sensitization to the subject of air quality in indoor environments, partially because some preventative measures introduced to reduce the risk of infection have provided for isolation of persons in closed spaces, whether these be workspaces or living spaces; in this way, with increased presence in indoor environments, the concentration of pollutants and the risk of virus transmission are increased.

It is further found that viruses are one of the most common causes of infective diseases transmitted in indoor environments, above all because of their features of high contagiousness and environmental resistance. However, it is also true that research on them in indoor environments, in the air and on surfaces, is not routinely pursued because of the wide variety of virus groups in existence, the major differences between them in terms of virulence and pathogenicity, and above all the lack of standard protocols for detecting them.

Recently, various measures and arrangements for sanitizing environments, aimed at containing human contagion, have been introduced for containing and managing the epidemiological emergency.

ISS COVID-19 Report No. 25/2020 expressly states that “where reference is made to sanitization, including in relation to the standards in force, this means the set of procedures and operations for cleaning and/or disinfection and maintenance of good air quality”.

Whilst some actions have been demonstrated and suggested for periodic sanitization of spaces through the use of technologies using ozone, chlorine, hydrogen peroxide or UVC rays, which can only be implemented in the absence of persons and by staff qualified in the use of these technologies, and products have been indicated for cleaning, sanitization and disinfection of surfaces, as regards the maintenance of good air quality all of the arrangements have been limited to indicating periodic changing (renewal) of air, along with some rules for the use of air-conditioning installations.

In particular, it is found that air renewal normally takes place by replacement with air drawn from the exterior by way of simple operations such as opening windows or activating mechanical ventilation systems which make it possible to filter or treat the air thermally or hygrometrically prior to admission into the environment.

However, filtration systems based on the photocatalytic properties of particular semiconductor materials are also available.

Photocatalysis is the natural phenomenon whereby a substance known as a photocatalyst affects the rate of a chemical reactor under the action of (natural or artificial) light.

More specifically, under the effect of light radiation of an appropriate wavelength, photocatalysts produce highly reactive species capable of decomposing the organic molecules present in the surrounding environment. The semiconductors owe the photocatalytic properties thereof to the reduced energy gap or band gap (E_{g}), which corresponds to the energy difference between the valence band and the conduction band. In general, insulators have an E_{g} greater than 4 eV, conductors are characterized by substantially zero E_{g} values, and semiconductors have E_{g} values less than 4 eV. It should be noted that, for efficient use of visible light as a source of radiation capable of inducing photocatalysis, the E_{g} value has to be around 2.0 eV. When the semiconductor is struck by radiation having energy greater than the associated E_{g}, an electron is promoted from the valence band to the conduction band and, in conjunction with the positive electron hole that is formed, generates a potential capable of oxidizing and/or reducing molecules adsorbed onto the surface of the photocatalyst. By way of example, if the photocatalyst is in contact with water molecules, the process may produce the hydroxyl radical (OH°) by oxidation and the superoxide anion (O₂⁻) by reduction. These highly reactive species thus compete to break down other organic or inorganic substances present in the environment surrounding the photocatalyst.

As a result of these photocatalytic properties, semiconductors have a wide range of applications both in the field of chemistry, where they can be used to break down environmental organic and inorganic pollutants into non-toxic, inert molecules, and in the field of microbiology, in view of the high antimicrobial activity provided by the radical oxygen species (ROS) produced by the photocatalyst.

Traditionally, the materials used in commercially available systems need ultraviolet light (UV of a wavelength less than 400 nm). The majority of the solutions on the market actually use photocatalysts based on titanium dioxide (TiO₂), which itself needs to be exposed to UV light, in particular UV-A, around 370 nm in order to be activated.

Italian patent application 102017000109448 describes a sanitizing photocatalytic reactor which uses tungsten trioxide (WO₃) as a semiconductor material acting as a photocatalyst. Tungsten trioxide has outstanding photocatalytic properties, which are primarily attributable to an E_{g} value less than that of other commonly used semiconductors, and has the advantage of being activated by natural light, in other words light falling within the frequency spectrum of visible light, unlike the traditionally used materials, which need a UV light. This is a clear advantage in terms of construction and operation. However, the solution according to patent application 102017000109448, while providing improvements over the previously known prior art (in particular because of the activation of the photocatalytic element which takes place without the need for a UV source), does not ensure a sufficient decrease in some harmful substances, in particular in relation to the elimination of viral agents and microorganisms.

The object of the present invention is to provide a technology which overcomes, at least in part, the drawbacks of the currently available systems.

### SUMMARY OF THE INVENTION

This result is achieved in accordance with the present invention by implementing a filter for air or fluids or aeroliquids, comprising: at least one photocatalytic element having a grid (or mesh) support covered at least in part by a layer of material comprising a solution based on a photocatalytic semiconductor, which can be activated by exposure to light radiation having a frequency in the visible spectrum; a plurality of LED light sources arranged so as to illuminate, when in use, at least one face of the at least one photocatalytic element; at least one filter element comprising a support of fabric made of hydrophilic material, impregnated with a solution of copper nanocluster. The photocatalytic semiconductor material preferably comprises tungsten trioxide (WO₃).

In a preferred embodiment of the present invention, the solution for the layer covering the grid support of the at least one photocatalytic element comprises one or more doping substances selected from: platinum, silver, vanadium. The grid support comprises a plurality of meshes or openings for the passage of air or fluids, the size of the meshes or openings being between 0.5 mm and 4 mm, preferably between 1 mm and 3 mm. In the present description we use the term mesh to indicate both the whole support (as alternative to “grid”) and the single opening included in the grid.

In a preferred embodiment, the plurality of light sources is arranged so as to illuminate, when in use, both sides of the photocatalytic element (or plurality of elements), although it is possible for only one of the sides of one or more elements to be illuminated.

One possible embodiment of the present invention may comprise a plurality of photocatalytic elements and a plurality of filtering elements with fabric impregnated with copper nanoclusters, in alternation with each other.

The grid support of the at least one catalytic element may take on various shapes, sizes and positions: it may for example be a substantially flat grid or a wavy grid. Another possible alternative solution for the grid support is for it to comprise a plurality of independent modules arranged substantially parallel to each other so as to form channels, the distance between the modules being between 0.5 mm and 4 mm, preferably between 1 mm and 4 mm.

The filter described above may take on various shapes: it may be squared shape, (e.g. substantially parallelepiped-shaped) or have a substantially toroidal shape in which the at least one photocatalytic element and the at least one filter element impregnated with copper nanocluster are concentric with each other.

In a preferred embodiment, the volume percentage of copper nanocluster, with respect to the support of fabric, is between 0.1% and 5%, preferably between 0.5% and 1.5%. According to the present invention, a system for sanitizing indoor environments is further implemented, comprising: the filter described above and a support structure that contains and keeps in position with respect to each other the at least one photocatalytic element, the plurality of LED light sources and the at least one fabric filter element impregnated with copper nanoclusters. The shape of the support structure may vary depending on the uses and functionalities of the filter: it may for example be a parallelepiped or a cylinder or has a toroidal shape.

In a preferred embodiment of the present invention, the technologies used for implementing the filter make it possible, by way of suitable air treatment devices, to sanitize the air (and other fluids) continuously even in the presence of persons, animals and foodstuffs so as to keep the quality thereof under control and reduce the effects of the presence of substances harmful to humans.

### BRIEF DESCRIPTION OF THE DRAWINGS

These and further advantages, objects and features of the present invention will be better understood by any person skilled in the art from the following description and the accompanying drawings, relating to example embodiments of an exemplary nature which are not to be understood as limiting, in which:

FIG. 1 shows a filter according to an embodiment of the present invention; the drawing includes three views of the same filter: FIG. 1a is a section of the filter; FIG. 1b is a perspective view; FIG. 1c shows the various elements separated from each other;

FIGS. 2, 3 and 4 schematically show possible alternative embodiments of the present invention. In each drawing, the view denoted “a” is a section of the filter; view “b” is a perspective view; view “c” shows the various elements separated from each other.

### DETAILED DESCRIPTION OF EMBODIMENTS OF THE INVENTION

The filter according to the present invention is based on the combined action of two materials designed appropriately for their use: tungsten trioxide (WO₃), used as a base material for implementing the photocatalytic reactor, and a solution of copper (Cu) nanocluster. In a preferred embodiment of the present invention, these materials are applied to appropriate supports (filters), one of a mesh/grid made of metal (or other material, e.g. plastics material) for the photocatalyst and the other of fabric made of cotton (or other hydrophilic material), which make it possible to sanitize the fluids when used in systems for air treatment (devices which take air from the environment and/or from the exterior, filter it, and reemit it after passage through the filtration system in question) and/or water filtration.

In the face of the health emergency challenge described above and the filtration capacity in terms of eliminating and/or inactivating a wide range of harmful substances present in the air in the confined elements, which current commercially available products have difficulty in eliminating without generating residues or requiring particular maintenance operations and/or replacements of parts or use of consumable materials, the present invention provides significant efficiency and is an important innovation, including on the basis of the efficacy tests performed in this period of health emergency, including on the infective virus COVID-19.

This mode of filtration differs significantly from the currently known techniques, which take place primarily on the basis of a single mechanical process (for example using HEPA filters) or a single chemical process using for example a photocatalyst. Virtually all of the known chemical solutions use, as a photocatalyst, titanium dioxide, or TiO₂, which has an E_{g} value of 3.6. Our solution, meanwhile, combines the action of two types of chemical process: one based on the action of a photocatalyst, the other on the quality of the carbon nanocluster. In addition, as a photocatalyst, a semiconductor activatable by natural light is used: in the preferred embodiment of the present invention, the photocatalyst is based on tungsten trioxide WO₃, which has an E_{g} value of 2.6. This lower E_{g} value makes it possible for the photocatalyst to be activated by lower-frequency light rays, and thus does not require light in the UV spectrum for activation.

In a preferred embodiment of the present invention, the use of the filter in forced ventilation systems causes the air to skim the surfaces of the support - as is described in greater detail below - so as to give rise to a series of chemical reactions in very narrow timeframes, on the order of billionths of seconds, capable of producing hydroxyl radicals (or hydrogen peroxide and/or ROSs) capable of attacking and disrupting, at the molecular level, the organic and inorganic substances, such as viruses, bacteria, fungi, moulds, odours, fine powders and volatile organic compounds (VOCs), present in the air. The further antibacterial and antiviral activity of the copper ensures significant results for the quality of the air, especially for certain pathogens (bacteria and viruses).

As regards the photocatalysis, the chemical reactions in question take place as a result of the energy that a small source of illumination, positioned close to the catalysing support, transfers by way of its own photons to the catalyst, creating electron holes thereon which combine with the water present in the air in the form of vapour, thus giving rise to the hydroxyls and the other oxidizing substances mentioned above.

For the purpose of sanitizing the air, the device according to the invention uses the combined effect of a high-efficiency nanoparticulate catalyst, based for example on tungsten trioxide (WO₃), and the copper (nanocluster) solution. The combined effect of the two components gives a result which is higher than the simple addition of the two separate actions, creating a sort of synergy which improves the total sanitizing effect. As shown for example in the Test made by the Viral Pathogenesis and Biosafety Unit of the San Raffaele Hospital of Milan on the viral strain SARS-CoV-2, the time to totally eliminate (or inactivate) the Virus was sensibly less than expected. A mention of the results can be found at the end of the present description and they were also published in an extended form on the web at
https://nanohub.it/wp-content/uploads/2021/06/NANOHUB-report-S.Raffaele Sars-CoV-2 ITA.pdf
   see also English version:
https://nanohub.it/wp-content/uploads/2021/06/NANOHUB-report-S.Raffaele Sars-CoV-2 translated-EN.pdf

Another source of information and of test results is the article “Rapid Inactivation of SARS-CoV-2 by coupling Tungsten Trioxide (WO3) Photocatalyst with Copper Nanoclusters”, Journal of Nanotechnology and Nanomaterials. 2020, Volume I, Issue 3, pages 109-115; we note that one of the author of such article is mentioned as the main inventor in the present patent application. With respect to the other solutions on the market, mechanical filters or the use of more commonly commercially available photocatalytic catalysts (such as the more widespread titanium dioxide, TiO₂), as a whole the use of WO₃ and of the combined action of copper renders the filter highly efficient in performing its sanitizing function. Indeed, WO₃ is more sensitive to electromagnetic radiation than the alternative catalysts, and therefore requires less energy in order to give rise to the oxidation reactions (and thus a small source of light, which is moreover in the visible spectrum, such as an LED light, is sufficient to activate the catalyst), while the copper increases its antibacterial and antiviral capabilities.

The filter does not lose its efficacy over time, since the WO₃ recovers the electron holes still in contact with the air skimming the surfaces treated therewith, and thus returns to its initial state, while the copper maintains its antiviral and antibacterial capacities.

A further important feature of the filter is that the WO₃ does not decompose over time, giving rise to dangerous substances, and that the result of the photocatalysis process, in other words of the decrease in pollutant and pathogenic elements, simply gives rise to substances that are harmless to breathe, attributable to low concentrations of water vapour, carbon dioxide, and sodium carbonates and nitrates, and the copper remains strongly adherent to the fabric support thereof.

This feature renders the filtration system suitable for use in all environments, in a continuous mode of operation, and even in the presence of persons, animals and/or foodstuffs, thus resolving one of the main aspects relating to sanitization of environments, which as noted above involves keeping the good air quality constant even in the presence of continuous contaminating effects. A further possible application of the filter according to the invention is for the implementation of individual protection devices, such as personal masks.

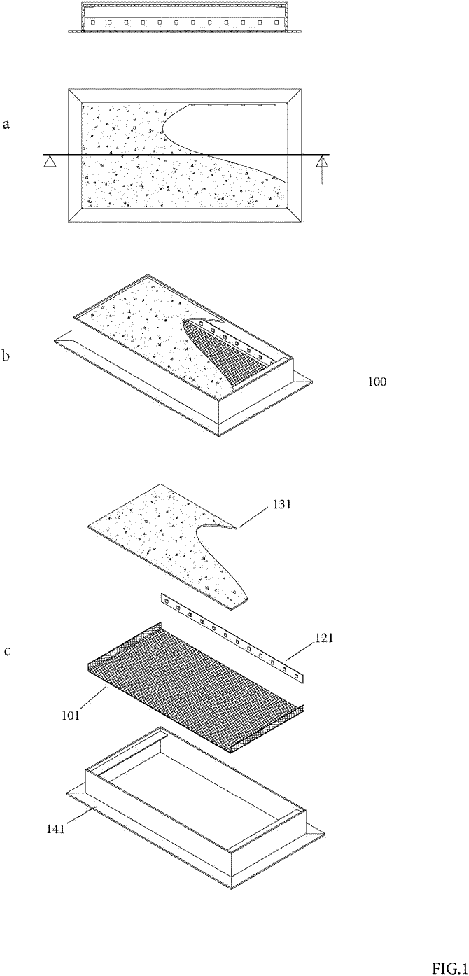

As is shown in FIGS. 1a, 1b and 1c, in particular in FIG. 1c, which shows the separate components, according to an embodiment of the present invention the filter comprises substantially 3 elements, which may be repeated to increase effectiveness and efficiency: A mesh (grid) filter, for example made of metal (although other materials are possible, for example plastics material, ceramic, glass fibre), of a variable geometry (101), in which the distance between the meshes is at least 0.5 mm and at most 4 mm, preferably between 1 and 3 mm. The filter may take on various shapes (depending on the use of the air treatment devices); in the embodiment shown in FIG. 1 it is a flat grid, but it may take on different shapes (wavy, pleated, etc.) so as to increase the air contact surface area while maintaining a reduced resistance against passing through. To this mesh (grid) filter, for example made of metal, the photocatalyst based on WO₃ is applied, so as to create the photocatalytic reactor of the filter. The photocatalyst may be applied in various ways, in accordance with the prior art, for example by immersion or spraying. In a preferred embodiment of the present invention, the photocatalyst is formed, in a liquid solution based on water and/or methanol, of tungsten trioxide (WO₃), platinum and alloys, by adhesion of the product to the surfaces. The function of the platinum is to increase the photocatalytic properties of the WO₃; other doping substances may alternatively be used, such as silver or vanadium.

An LED, e.g. strip LED, illumination system (121) makes it possible to illuminate both sides of the metal mesh/grid filter (101) so as to activate the photocatalytic reactor even in the visible light spectrum. The LED lights could also illuminate a single side of the photocatalytic element 101, but the efficiency of this would be limited.

A fabric 131, for example made of cotton (or another hydrophilic textile material) is impregnated with a copper nanocluster solution, which is described in the following.

In a preferred embodiment, the three elements mentioned above are housed in a container (141) which holds them together and makes it possible for air to pass through the two filters 101 and 131. This container may take on various shapes, depending on the use to which it is to be put. For a use in the context of a system for purifying/sanitizing indoor environments, the container will presumably be substantially parallelepiped-shaped, or else, as will be seen in a subsequent example, cylindrical, or better toroidal. The mesh/grid photocatalytic reactor, for example made of metal, could also be repeated two/three (or even more) times within said system, by virtue of the efficiency that it is desired to obtain in terms of the filter and in terms of the volume of air to be treated.

A configuration of the filter implemented on a suitable support structure could thus, for example, be:

fabric with copper nanocluster + LED illumination system + photocatalytic reactor having metal mesh/grid + LED illumination system + copper nanocluster fabric.

The dimensions of the filter may also be variable as a function of the application thereof to devices and/or installations for mechanical treatment of air, and even on the basis of the results which it is desired to obtain as regards the treatment time and the volume of air to be treated.

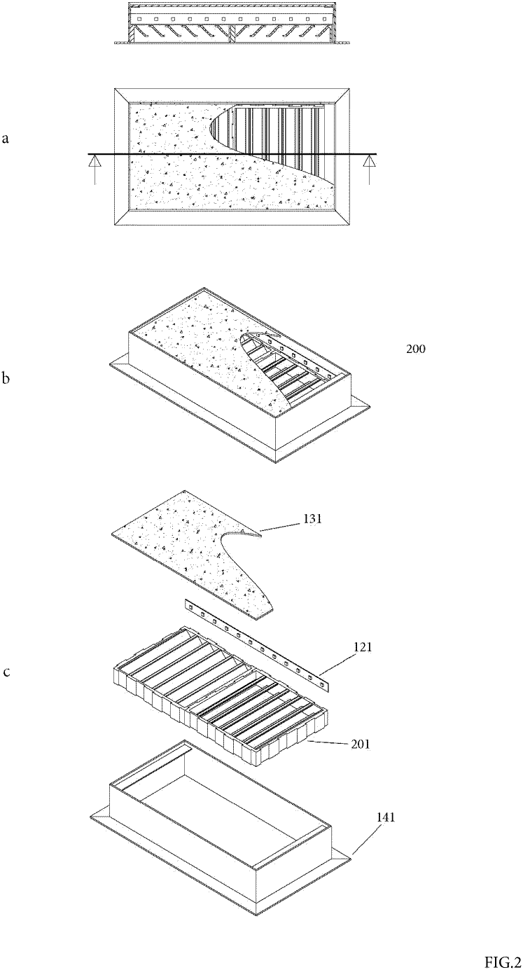

For greater efficiency, it is possible to arrange the element with the photocatalyst in a different geometry from the arrangement with a flat grid shown in FIG. 1. For example, the element could be arranged in a series of substantially parallel modules 201, as is shown in FIG. 2 (with the various views a, b and c). The LED illumination system 121 and the fabric impregnated with copper nanocluster 131 remain substantially unchanged from the configuration of FIG. 1. In this way, the exposed surface area of the photocatalyst is increased without substantially changing the overall dimensions of the filter 200. The distance between the various parallel modules is preferably between 0.5 mm and 4 mm, even more preferably between 1 mm and 3 mm.

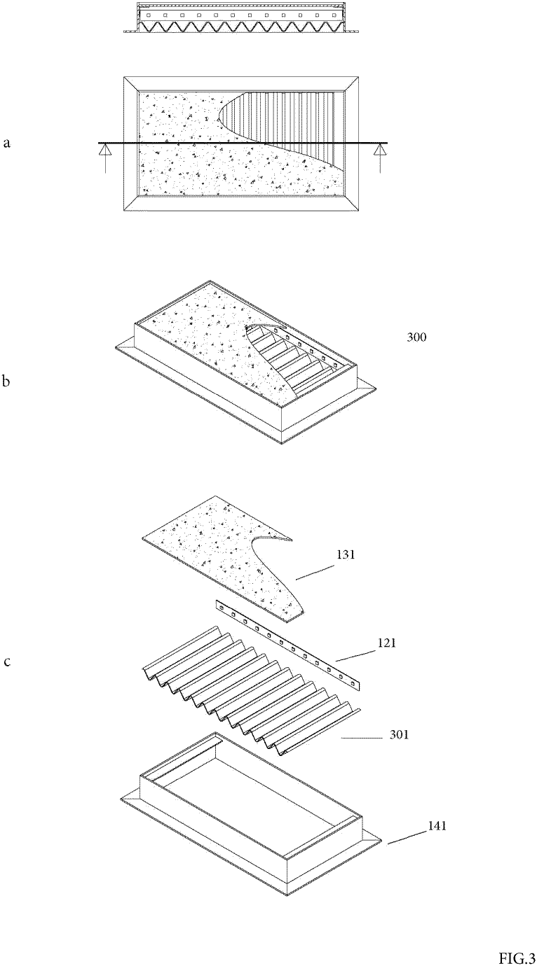

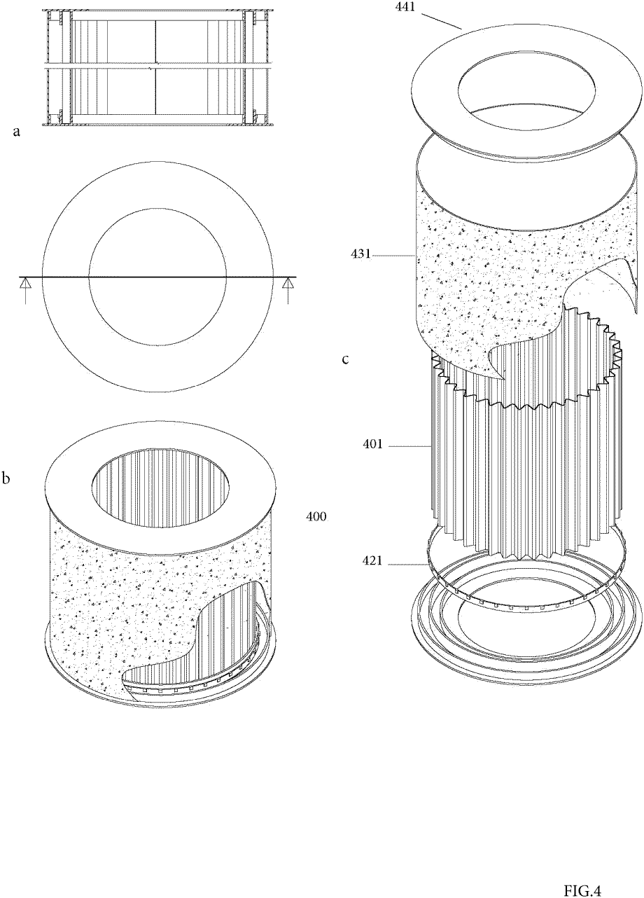

Another possible version of the filter (300) is shown in FIG. 3, in which the element with a photocatalyst is formed by a wavy grid 301. In this case too, the other components (the LED illumination element 121 and the fabric impregnated with copper nanocluster 131) remain substantially unchanged, as does the container 141. What does change is the geometry of the support for the photocatalyst, which, both in this case and in the example of FIG. 2, provides a larger exposed surface area than the configuration of FIG. 1. Meanwhile, FIG. 4 (with its views a, b and c) shows an alternative implementation of the filter, which in this case takes on a cylindrical or more precisely toroidal shape. In this case, the photocatalytic element 401 is arranged, in a substantially cylindrical shape (in a wavy configuration in this case, although other solutions, including the flat and modular ones shown previously, are possible), within the element with impregnated fabric 431. Between the two cylindrical elements, the LED illumination system 421 is placed, which in this case is a circular LED strip. The container 441 will be appropriately positioned for holding together the aforementioned components. In this case, as will be immediately clear to persons skilled in the art, the passage of the fluid to be sanitized (e.g. air) will have to take place by way of introduction from one of the two ends and expulsion via passing through the two (or more) filter elements.

In the configuration shown in FIGS. 2, 3 and 4, too, repetitions of elements are possible for increasing the treatment capacity of the filter for the fluid (e.g. air), as discussed previously with reference to FIG. 1.

### Photocatalytic Element

Among the metal oxides used as photocatalysts, tungsten trioxide (WO₃) is taking on a more and more important role because of the outstanding photocatalytic properties thereof, which are primarily attributable to a lower E_{g} value than other commonly used semiconductors and which render it active in the visible light range.

The development of a novel photocatalyst based on WO₃ has thus significantly increased the efficacy of the photocatalyst and eliminated the problem of the use of UV light.

The photocatalysis mechanism in the presence of WO₃ is thus similar to that which occurs with TiO₂, but in the presence of a different radiating light (visible light) having a wavelength greater than 400 nm: when exposed to light in the visible spectrum, the WO₃ absorbs and converts the light energy into electrons and electron holes. The WO₃ reacts with water (e.g. moisture in the air) to create hydroxyl radicals (expressed as OH⁻) and with oxygen to create superoxide anions (O₂⁻); billions of particles (ions or radicals) of these highly oxidizing species are created in billionths of a second, and work to disrupt the material at the molecular level. This results in effective decomposition of the organic and inorganic polluting substances of nitrogen oxides, polycondensed aromatic compounds, sulphur dioxide, carbon monoxide, formaldehyde, methanol, ethanol, benzene, ethylbenzene, etc. and in an effect of inactivating and decomposing simple or complex microorganisms (viruses, bacteria).

The strong oxidizing effect makes it possible to use the photocatalyst based on WO₃, tungsten trioxide, as a photocatalytic disinfectant.

Although many studies on the photocatalytic inactivation of bacteria have been recorded, few studies have dealt with the inactivation of viruses.

The studies on the transformation of viruses by photocatalysis have been carried out in an aqueous or other liquid environment or else by a method of direct organism/surface contact, and there are two levels of photocatalytic attack:
photoinactivation or photodeactivation, with a resulting disinfectant effect;
decomposition/killing-off of the viral cells, with a resulting sterilizing effect.

The mechanism of virus inactivation by photocatalysis is yet to be definitively explained, but the effectiveness of the system has already been demonstrated by laboratory tests, using numerous types of microorganisms and also with quantification of the virtually complete result of the attack.

This seems to be undertaken on the virus particles by way of adsorption of said particles onto the surfaces of the catalyst, followed by an attack on the protein capsid and on the bonding sites of the virus (direct redox attack). According to other sources, the inactivation behaviour of the virus is brought about by hydroxyl radicals •O₂⁻ and OH• or else (in addition) by reactive oxygen species (ROSs) such as •O₂⁻, OH⁻ H₂O •HO, which are free in the bulk phase, and not by those bonded to the surface of the catalyst. The mechanism of subsequent decomposition involves breakdown of the cell wall and cytoplasmic membrane, again caused by the production of reactive oxygen species (ROSs). This initially leads to the expulsion of the cell content, and thus to cell lysis, to the point of complete mineralization of the organism. The closer the contact between the virus and the catalyst, the more effectively it is killed off.

Whilst the ambient conditions at the interface should be taken into account, the reactive species have an action radius of up to 2 mm from the active surface.

For this reason (action radius), in a preferred embodiment of the present invention the photocatalytic reactor comprises a metal mesh (grid) fabric which makes adhesion of the photocatalyst possible along with a distance between the catalysed surfaces of between 0.5 mm and 4 mm, preferably not more than 3 mm. The grid/mesh support may also be implemented using other materials, for example plastics material, glass fibre, as long as it ensures a sufficient support for the photocatalyst.

This sanitizing photocatalytic reactor is suitable in the presence of aeroliquid or liquid fluids, and basically comprises a reaction zone, to which the nanometric, natural light photocatalyst based on WO₃ is applied, and a zone of illumination by white LED lights, which activates the photocatalyst. In a preferred embodiment of the present invention, the fluids to be sanitized skim the reaction zone and are thus subjected to a photocatalytic decomposition treatment.

As is shown in detail in FIGS. 1-4, without prejudice to the structural differences between various implementations, the sanitizing photocatalytic reactor according to the present invention for aeroliquid or liquid fluids comprises:
a reaction zone having a metal mesh/grid (inert support) of variable geometry in which the meshes, which are not spaced more than 4 mm apart, preferably 3 mm apart, from each other, are covered by a layer of nanotechnological material which is photocatalytic in natural light and is based on WO₃;
a zone of illumination by LED lights, preferably white, so as to illuminate the surfaces of the metal mesh/grid support in such a way that each portion or, if present (for example in the embodiment shown in FIG. 2), each channel is illuminated by the LED light.

In a preferred embodiment of the present invention, the inert metal mesh/grid support, to the surface of which the photocatalyst is applied, is formed from steel or other metal alloys (for example aluminium or copper). Alternative materials may be plastics material, ceramic, and glass fibre. The dimensions of the metal mesh/grid support are based on the dimensions of the application to be implemented, for both the length and the height (total surface area); meanwhile, the thickness may be variable, including in relation to the volumes of fluid to be treated, and may be formed by more mesh/grid sheets or by particular configurations of mesh.

The principle behind the selection and dimensioning is to optimize the surface area of the catalytic element exposed to the fluid to be treated, obtaining a uniform distribution of the fluid at the input and output, in such a way that it can be fully treated in the most effective system possible, while correct illumination of all of the contact surfaces is simultaneously ensured. It is important to verify, for the dimensioning, the flow speed of the (aeroliquid or liquid) fluid to be sanitized for determining the length dimension (along the air direction) of the metal mesh/grid, which must not be underdimensioned in terms of the ratio between reaction time and speed of passage. Both the reaction zone and the supply zone may be parts of machines or apparatus which aspirate fluids.

In the following, the results are set out of a test carried out to verify the decrease in airborne contamination with a ventilator on the metal mesh reactor with adhesion of the photocatalyst based on WO₃ and activated by LED light; the test was carried out in accordance with the following reference standards:

total bacterial load at 22° C. (reference method: standards M.U. 1962-1:06 and UNI EN ISO 6222:2001)
total bacterial load at 37° C. (reference method: standards M.U. 1962-1:06 and UNI EN ISO 6222:2001)
moulds and yeasts (reference method: standards M.U. 1962-1:06 and ISTISAN REPORTS 2007/5 PAGE 164 MET ISS A 016B
Escherichia coli (method: M.U. 1962-1:06 and APAT CNR IRSA 7030 F Man 29 2003)

in accordance with the following operating procedure:

1) In the space identified as a reference environment (5 m³) for carrying out the test, the apparatus to be verified was arranged, which is referred to as an “aspirator” and in the interior of which a metal grid was attached, which was treated with WO₃ and had the function of a photocatalytic support (dimensions 50 cm × 20 cm).

The sampling was carried out using an instrument referred to as a bio-sampler, consisting of a test tube containing sterile water in which the aspirated air to be tested was bubbled, via a specific external pump, so as to transfer the bacterial load present in the air to the sterile liquid contained in the test tube.

2) 4 samplings were carried out, each of a duration of 30 minutes.
a) The first test (control) related to the sampling of the air in the test environment to test the base bacterial load used so as to verify any decrease in the subsequent tests; thus, the ambient sample was not in any way subjected to sanitization treatment - untreated air - zero point;
b) The second sampling was performed after initial contamination by sampling the ambient air with a deactivated photocatalytic system;
c) The third sampling was performed after the first sanitization treatment by sampling the ambient air after photocatalytic treatment activated for 60 minutes;
d) The fourth and final sampling was performed after a second sanitization treatment, to verify the ambient air after photocatalytic treatment activated for 120 minutes.

The results of the test are set out in the tables below:

| SAMPLING | BIOLOGICAL PARAMETERS | RESULT cfu/m³ | METHOD OF ANALYSIS | Detection limit |
|---|---|---|---|---|
| ZERO-POINT CONTROL | Total bacterial load at 22° C. | 182 | Standards M.U. 1962-1:06 and UNI EN ISO 6222:2001 | 10 |
| Untreated air | Total bacterial load at 37° C. | 109 | Standards M.U. 1962-1:06 and UNI EN ISO 6222:2001 | 10 |
| | Moulds and yeasts | 25 | Standards M.U. 1962-1:06 and ISTISAN REPORTS 2007/5 PAGE 164 MET ISS A 016B | 10 |
| | Escherichia Coli | <10 | Method M.U. 1962-1:06 and APAT CNR IRSA 7030 F Man 29 2003 | 10 |

| SAMPLING | BIOLOGICAL PARAMETERS | RESULT cfu/m³ | METHOD OF ANALYSIS | Detection limit |
|---|---|---|---|---|
| | Total bacterial load at 22° C. | 255 | Standards M.U. 1962-1:06 and UNI EN ISO 6222:2001 | 10 |
| INITIAL CONTAMINATION | Total bacterial load at 36° C. | 300 | Standards M.U. 1962-1:06 and UNI EN ISO 6222:2001 | 10 |
| Untreated contaminated air | Moulds and yeasts | 100 | Standards M.U. 1962-1:06 and ISTISAN REPORTS 2007/5 PAGE 164 MET ISS A 016B | 10 |
| | Escherichia Coli | <10 | Method M.U. 1962-1:06 and APAT CNR IRSA 7030 F Man 29 2003 | 10 |

| SAMPLING | BIOLOGICAL PARAMETERS | RESULT cfu/m³ | PERCENTAGE DECREASE FROM INITIAL CONTAMINATION | METHOD OF ANALYSIS | Detection limit |
|---|---|---|---|---|---|
| 1^{ST} TREATMENT Air | Total bacterial load at 22° C. | 55 | 79% | Standards M.U. 1962-1:06 and UNI EN ISO 6222:2001 | 10 |
| contaminated and treated for 60 minutes | Total bacterial load at 36° C. | 70 | 76% | Standards M.U. 1962-1:06 and UNI EN ISO 6222:2001 | 10 |
| | Moulds and yeasts | 10 | 90% | Standards M.U. 1962-1:06 and ISTISAN REPORTS 2007/5 PAGE 164 MET ISS A 016B | 10 |
| | Escherichia Coli | <10 | / | Method M.U. 1962-1:06 and APAT CNR IRSA 7030 F Man 29 2003 | 10 |

| SAMPLING | BIOLOGICAL PARAMETERS | RESULT cfu/m³ | PERCENTAGE DECREASE FROM 1^{ST} TREATMENT TEST (%) | PERCENTAGE DECREASE FROM INITIAL CONTAMINATION (%) | METHOD OF ANALYSIS | Detection limit |
|---|---|---|---|---|---|---|
| | Total bacterial load at 22° C. | 20 | 36% | 92% | Standards M.U. 1962-1:06 and UNI EN ISO 6222:2001 | 10 |
| 2^{ND} TREATMENT Air contaminated and treated for 120 minutes | Total bacterial load at 36° C. | 15 | 21% | 95% | Standards M.U. 1962-1:06 and UNI EN ISO 6222:2001 | 10 |
| | Moulds and yeasts | 10 | / | / | Standards M.U. 1962-1:06 and ISTISAN REPORTS 2007/5 PAGE 164 MET ISS A 016B | 10 |
| | Escherichia Coli | <10 | / | / | Method M.U. 1962-1:06 and APAT CNR IRSA 7030 F Man 29 2003 | 10 |

From the final evaluation of the analyses performed under the various operating conditions, it can be found, as shown above, that there is a result in line with expectations for the ambient conditions at time zero.

The situation changes greatly, however, after the treatments of the air with the photocatalytic system after an hour of treatment (namely recycling the same air within the environment), and even more greatly after two hours of treatment.

In practice, a considerable decrease is already found after the first hour of operation, whilst in the second hour yields greater than 90% are reached over all of the researched biological indicators.

### The Cotton Fabric Filter Carrying Copper Nanocluster

As is known, copper is a metal having exceptional thermal exchange and electrical conductivity properties. In addition, copper is one of the few materials which can be recycled continuously while keeping the original physical and technological features thereof constant. Copper is also present in food, and is necessary for our metabolism, and needs to be present in our diet.

Copper has recognized capabilities in inactivating or eliminating bacteria, microbes, moulds, fungi and viruses. In particular, these capabilities of copper have been demonstrated in the literature, deriving from scientific research performed on various types of pathogenic agents such as: Acinetobacter baumannii, Adenovirus, Aspergillus niger, Candida albicans, Campylobacter jejuni, Clostridium difficile, Enterobacter aerogenes, Escherichia coli (strain O157:H7), Helicobacter pylori, Influenza A (H1N1), Legionella pneumophilia, Listeria monocytogenes, MRSA (including E-MRSA), Poliovirus, Pseudomonas aeruginosa, Salmonella enteriditis, Staphylococcus aureus, tuberculosis bacilli, VRE (Vancomycin-resistant enterococcus).

To implement one of the essential elements in accordance with a preferred embodiment of the present invention, a filter of fabric made of synthetic and/or natural fibres was produced, functionalized with submicronic copper cluster.

The method for coating the fibres and/or fabrics is found to be eco-friendly, and does not lead to emissions of toxic or environmentally polluting elements. Specifically, it is a system of impregnation in aqueous solution at room temperature.

Various amounts of Cu nanoparticles (on the order of 10% of the overall weight of the fabric) were deposited on the cotton fibres. The dimensions and the surface density of the deposited clusters may be varied and controlled in a wide range, bringing about variable properties and appearance. In particular, the interval between 20 and 400 nm represents one claim of the present invention.

The clusters are synthesized from an aqueous solution based on copper salt, by a controlled process of chemical reduction of the previously solubilized copper salts.

The solution is matured in approximately 24 hours from the solubilization of the precursors.

The appropriately matured solution is used to impregnate a fibre fabric, which is used as an air filtration system with the function of retaining particles and pathogens.

Typically, the salt used is Cu acetate solubilized in water.

Hydrazine hydrate is gradually added as a reducing agent. Then, the cotton gauze fabric was immersed in the solution and mixed so as to absorb all the copper.

After the synthesis, the cotton gauze was washed several times with distilled water and dried in a furnace at 65 degrees centigrade; the thermal treatment makes it possible to evaporate the solvent completely and provide strong adhesion of the cluster to the cotton fibres.

The antibacterial activity was tested against gram-negative E. coli bacteria by the halo test method, which showed a good antibacterial effect of the fabrics coated with Cu nanoparticles.

### Efficiency of the Filter According to an Embodiment of the Present Invention

This filtration system (metal grid with adhesion of the photocatalyst based on WO₃ and copper nanocluster fabric) was tested at the Viral Pathogenesis and Biosafety Unit of the San Raffaele Hospital of Milan on the viral strain SARS-CoV-2 to verify the effectiveness of inactivation on the infective virus.

So as to be able to perform the test in liquid solution, two devices were provided, into which were inserted the photocatalytic reactor having a metal mesh/grid and the fabric having copper nanocluster, into which the liquid with infected cells was to be inserted and was to be removable to verify the result.

The test was performed twice (to confirm the results), with a sample after treatment for 3 periods of time (10, 30 and 60 minutes) the first time and for 4 periods of time (10, 20, 30 and 60 minutes) the second time.

The experiment gave positive results, demonstrating partial inactivation at 10, 15, 20 minutes and total inactivation at 30 minutes and one hour.

The potential residues of virus were also measured after eliminating all of the first volume of 80 ml which contained the virus, and no trace of virus remained.

In all the above examples, reference was always made to tungsten trioxide (WO₃) as the material semiconductor forming a basis for the photocatalytic layer. As will be appreciated by persons skilled in the art, other materials having similar activation features (in other words activatable by exposure to a radiation having a frequency within the visible light spectrum) could alternatively be used.

In practice, the details of implementation may in any case vary in an equivalent manner as regards the individual structural elements described and illustrated and the nature of the indicated materials, without thereby departing from the adopted solution idea and while remaining within the limits of the teaching provided by the present patent. A person skilled in the art may make many alterations to the solution described above so as to meet local or specific requirements. In particular, it should be clear that, although implementation details based on one or more preferred embodiments have been provided, omissions, replacements or variations of some specific features or some steps of the described method may be applied with a view to design or implementation requirements.

1. Filter for air or fluids or aeroliquids, comprising:
at least one photocatalytic element having a grid/mesh support covered at least in part by a layer of material comprising a solution based on a photocatalytic semiconductor, which can be activated by exposure to light radiation having a frequency in the visible spectrum;
a plurality of LED light sources arranged so as to illuminate, when in use, at least one face of the at least one photocatalytic element;
at least one filter element comprising a support of fabric made of hydrophilic material, impregnated with a solution of copper nanocluster.

2. The filter according to claim 1, wherein the photocatalytic semiconductor comprises tungsten trioxide.

3. The filter according to claim 2, wherein the layer covering the grid support of the at least one photocatalytic element comprises one or more doping substances selected from: platinum, silver, vanadium.

4. The filter according to claim 1, wherein the grid support comprises a plurality of meshes or openings for the passage of air or fluids, the size of the meshes or openings being between 0.5 mm and 4 mm.

5. The filter according to claim 1,
wherein the plurality of light sources is arranged so as to illuminate, when in use, both
sides of the at least one photocatalytic element.

6. The filter according to claim 1, comprising a plurality of photocatalytic elements and a plurality of filtering elements with fabric impregnated with copper nanoclusters, in alternation with each other.

7. The filter according to claim 1, wherein the grid support of the at least one catalytic element is a substantially flat grid.

8. The filter according to claim 1, wherein the at least one photocatalytic element comprises a plurality of independent modules arranged substantially parallel to each other so as to form channels, the distance between a module and the next being between 0.5 mm and 4 mm.

9. The filter according to claim 1, wherein the grid support of the at least one catalytic element is a wavy grid.

10. The filter according to claim 1, wherein the filter has a substantially toroidal shape in which the at least one photocatalytic element and the at least one filter element impregnated with copper nanocluster are concentric with each other.

11. The filter according to claim 1, wherein the volume percentage of the copper nanocluster, with respect to the support of fabric, is between 0.1% and 5% preferably between 0.5% and 1.5%.

12. An indoor sanitization system, including:
the filter of any preceding claims; and
a support structure that contains and keeps in position with respect to each other the at least one photocatalytic element, the plurality of LED light sources and the at least one fabric filter element impregnated with copper nanoclusters.
